(19) United States
(12) Patent Application Publication
(10) Pub. No.: US 2006/0167366 A1
(43) Pub. Date: Jul. 27, 2006

(54) Method and apparatus for extracting biological signal such as heartbeat or respiration

(76) Inventor: Seijiro Tomita, Komae-shi, Tokyo (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/548,058

(22) PCT Filed: May 7, 2003
(86) PCT No.: PCT/JP03/05711

§ 371 Date: Sep. 6, 2005

(51) Int. Cl. A61B 5/04

(52) U.S. Cl. 600/518

(57) ABSTRACT

The present invention relates to a signal detection and processing method and a device therefor designed for selecting a main periodic information from general time series signals (analog signals) involved in biological signals such as heart beat and breathing, wherein a heart rate, a respiratory rate and data fluctuations thereof are output by a sensor which detects integrated biological signals with periodicity such as heart beat and breathing of a human body.

### BACKGROUND OF THE INVENTION

1. Field of the Invention

The present invention relates to a signal detection and processing method and a device therefor designed for selecting a main periodic information from general time series signals (analog signals) involved in biological signals such as heart beat, and in particular, relates to a method and a device therefor in which a heart rate, a respiratory rate and the two data fluctuations are output by a sensor that detects integrated biological signals such as a heart beat and a breathing with periodicity of a human body.

2. Description of the Related Art

In a case in which a sensor or a measuring device detects a one-dimensional natural phenomena, the phenomena is often expressed in voltage level, but due to the periodical recurring many of the obtained data form a waveform when they are graphically expressed.

A method to calculate a waveform period by finding a peak and a method to track frequency components by the fast Fourier transformation (FFT method) have been known. “Living Signal Measuring Method” disclosed in Japanese Patent Disclosure No. 2003-061925 and “Pulse Oximeter Using a Virtual Trigger for Heart Rate Synchronization” disclosed in TOKUHYOHEI No. 10-510440 have been known as examples.

A method to calculate a waveform period by finding a peak of the waveform has been known. Problem with the method and measuring device is that a peak position and a peak height may always be fluctuated by an external factor such as noise.

In particular, another weak point with this method and measuring device is that as a respiratory signal has a very low frequency close to a direct-current component, it easily receives influences of dynamic range of amplification circuit or temperature behavior.

On the other hand, another method to calculate frequency components with the fast Fourier transformation (FFT method) has been known as a frequently used method.

But, a weak point for the conventional method described above is that since a vital data also involves high-frequency component such as a heart beat and a biological signal such as a breathing has a very low frequency, a big error is produced due to a low resolution of the frequency of the FFT method.

Another problem is that since the conventional art needs to process many calculations at a high speed in real time so as to detect all frequency components, the art requires a larger circuit and an increase in consumption currency. Thus, it was difficult to apply the conventional art to a compact portable device.

In view of the above problems in the conventional art, the present invention has as an object to provide a selection of biological signals such as heart beat and breathing and device therefor.

A first object of the present invention is to provide a selection of biological signals such as heart beat and breathing and device therefor which detects a waveform period in a stable performance without being influenced by a signal level or a peak height, desired for a compact size measuring device with low power consumption.

A second object of the present invention is to provide a selection of biological signals such as heart beat and breathing and device therefor which precisely detects a waveform period without having the waveform deformed by external factors such as a noise and without fluctuating a peak position.

A third object of the present invention is to provide a selection of biological signals such as heart beat and breathing and device therefor which can obtain a detection period extremely close to a visual waveform period.

### SUMMARY OF THE INVENTION

The present invention solves the above-mentioned problems with the following means.

A first aspect of the present invention provides a selection of biological signals such as heart beat and breathing according to the claim 1 of the invention, wherein:
forms of biological signals such as a heart beat and a breathing are modulated from time series signals (analog signals) into digital signals with an analog-to-digital modulation means;
a level data of the digital signal is the same;
a changing trend (upward trend and downward trend) of the digital signal is the same; and
most closer two points on an axis of time (hereafter referred to as “in-phase points”) are detected with an in-phase points detection means,
   and wherein after a distance between the two in-phase points (hereafter referred to as “running distance between in-phase points”) is detected with a detection means of a running distance between in-phase points, a detected information with the detection means of a running distance between in-phase points is computed with a statistical processing means, an information with the statistical processing means being analyzed with a measuring means for frequency of occurrence so as to calculate a frequency of occurrence of a same running distance, and a measurement result with the measuring means for frequency of occurrence being analyzed so as to output a most frequent running distance as a main period with a main period output means.

A second aspect of the present invention provides a selection device of biological signals such as heart beat and breathing according to the claim 2 of the invention, comprising:

an analog-to-digital conversion means which converts forms of biological signals such as a heart beat and a breathing from a time series signal (analog signal) to a digital signal;

an in-phase points detection means in which a level data of the digital signal has a same data, a changing trend (upward trend and downward trend) is the same, and in-phase points are detected on an axis of time;

a detection means of a running distance between in-phase points which detects a distance between two in-phase points;

a statistical processing means which computes the information that a detection means of a running distance between in-phase points detects;

a measuring means of frequency of occurrence which analyzes the information after computing and measures a frequency of occurrence of a same running distance;

a main period output means which analyzes a measurement result with a measuring means of a frequency of occurrence and outputs a most frequent running distance as a main period; and

a detection means of a waveform period of a biological signal in a stable performance without being influenced by a signal level or a peak height.

### BRIEF DESCRIPTION OF THE DRAWINGS

FIG. 1 is a drawing which illustrates a statistical analysis of a waveform signal that changes periodically.

FIG. 2 is a block diagram of algorithm according to the invention.

FIG. 3 is a flow chart for detection of in-phase points by tracking a waveform and a running distance therebetween.

FIG. 4 is an example of biological signals such as a heart beat and a breathing which are detected with a biological sensor.

FIG. 5 is an example of a statistical distribution, Cᵣ (t, y) and C_{d} (t, y), for a waveform of breathing.

FIG. 6 is a detected example of respiratory rate and heart rate.

### DESCRIPTION OF THE PREFERRED EMBODIMENTS

Now, embodiments of the present invention will be described by referring to the accompanying drawings.

In a case in which signals that occur periodically are analyzed with a statistical method, a cluster formation is observed in the statistical distribution.

The invention provides a concept of a running distance between in-phase points as well as a means of calculating a main period of a waveform with a statistical processing of the distance.

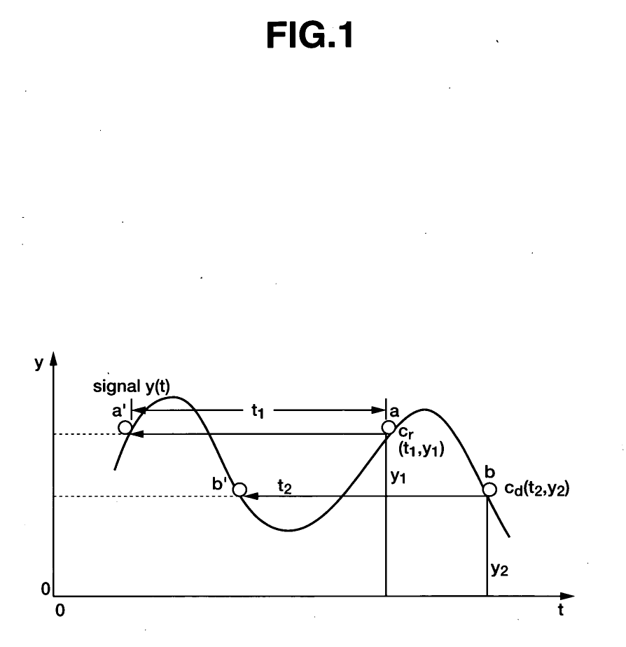

FIG. 1 illustrates a statistical analytic method for a waveform signal which changes periodically. In the figure, the horizontal axis, (t), shows an axis of time and the vertical axis, (y), shows a signal level. The graph shows a time series signal, y (t), which changes periodically.

As used herein, the term “in-phase points” shall mean two points, as shown above, which are at the same level, have a same change trend (upward or downward) and are most close to each other. In an example shown in FIG. 1, an in-phase point, a, is, a′, and an in-phase point, b, is, b′.

As used herein, the term “running distance between in-phase points” means a distance between two in-phase points which are located close to each other. In an example shown in FIG. 1, a running distance between the in-phase points, a, and, a′, is, t₁. A running distance between the in-phase point, b, and, b′, is, t₂.

CPU (Central Processing Unit) is used for statistical processing of a running distance between in-phase points. To facilitate it, analog waveform signals are modulated into digital waveform signal by an analog-digital modulation means.

The equation is shown as below.
y(tᵢ)tᵢ(iε{0,1,2, . . . , N−1}) Each sampling time
y(tᵢ)ε{0,1,2, . . . , Y} Signal value at each sampling time

Time and signal level appear as a discrete value.

As used herein, mathematical signs shown in equations of the invention shall mean the following:
y (i): Input waveform signal value at time, i
N: Number of waveform signal data sampled
Cᵣ (j, k) Buffer which stores statistical information concerning a frequency of the running distance, j, at two in-phase points at the same level, k, in an upward waveform.
C_{d} (j, k) Buffer which stores statistical information concerning a frequency of the running distance, j, at two in-phase points at the same level, k, in a downward waveform.
Cᵣ (t) Buffer which stores statistical information concerning a total frequency of the running distance, t, in an upward waveform.
C_{d} (t) Buffer which stores statistical information concerning a total frequency of the running distance, t, in a downward waveform.
Δt Detected error of a main periodic data

Many in-phase points are located in a waveform. In order to process a running distance between in-phase points statistically, in-phase points on the waveform are tracked.

First, signal value, {y (tⱼ)}, of input time series signal at each time is tracked from, t₀, sequentially.

For example, if a point on a waveform comes to a point, a (t₀+t₁, y₁), in an upward trend waveform, CPU finds the most close two points in the tracked waveform points which are at the same level and have a same change trend (upward).

At this time, a first cross point, a′ (t₀, y₁), where a line is drawn parallel to the axis of time in the direction to the starting point of the axis must be an in-phase point thereof. On the other hand, a point in a downward trend waveform can be also processed in the same manner.

All running distances between in-phase points on a waveform are statistically processed so as to detect a main period of a waveform.

For N−1 sampling data, after in-phase points are tracked in this manner, running distances between in-phase points are analyzed so as to process a frequency of occurrence of each running distance statistically. The frequency of occurrence is shown as Cᵣ (t, y) and, C_{d} (t, y).

Here, Cᵣ (t, y) shows a frequency of occurrence of an event where the signal level axis, y, intersects with the running distance, t, during the upward trend. C_{d} (t, y) shows a frequency of occurrence of the event where the signal level axis, y, intersects with the running distance t during the downward trend.
∀tε[0,To] and ∀y(t)ε[0,Y]

After the frequency of occurrence of the above running distance in the sampling interval has statistically processed, the following statistical calculation is performed.

$$\begin{matrix}{C}_{r} ( t ) = {\sum }_{y = 0}^{Y} {c}_{r} ( t , y ) & Equation 1 \\ {C}_{d} ( t ) = {\sum }_{y = 0}^{Y} {c}_{d} ( t , y ) & Equation 2\end{matrix}$$

Parameters which relate to a signal level of the frequency of occurrence are reduced by arithmetic addition to draw only a running distance, one-dimensional variable, Cᵣ (t) and C_{d} (t).

According to the above determined one-dimensional variables, Cᵣ (t) and C_{d} (t), a main period data of the waveform can be derived by the following equation.

$$C = {max}_{t} ( {\sum }_{t = T}^{T + δ t} {C}_{r} ( t ) + {C}_{d} ( t ) ) \\ ∀ t ∈ [ {T}_{1} , {T}_{2} ] \qquad Equation 3$$

A main period data, T, as a time series signal, y(t), is derived by the following determination rule.

If a equation of C> a certain threshold level is true, a main period data is determined to be T. In this equation, δ t is set within the limits of what is allowed for detection error of the period data.

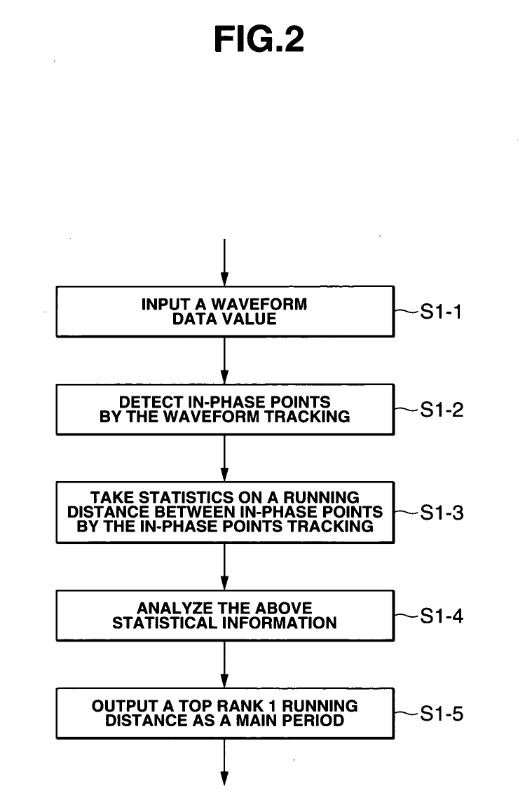

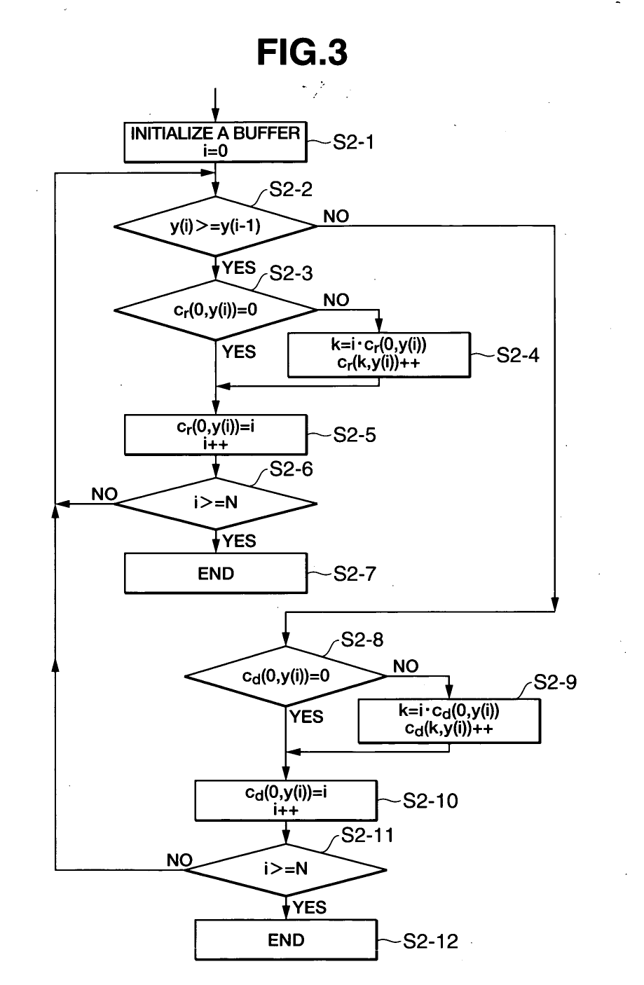

FIG. 2 is a block diagram showing algorithm of the invention. FIG. 3 relates to the algorithm in the step S1-2 of the FIG. 2, in which a flow chart illustrates a detection of in-phase points by tracking a waveform and a running distance.

First, output analog signals from the detector or the sensor are sampled at a sampling interval. The sampled signals are modulated into digital signals by an analog-digital modulation means.

As shown in FIG. 2, the obtained digital signal data are fed into the step S1-1. In the step S1-2, in-phase points are tracked so as to process a running distance between in-phase points statistically.

Through these processes, a running distance, Cᵣ (t, y), and a frequency of occurrence, C_{d} (t, y), are calculated for in-phase points on a waveform at each instant of time.

Next, according to the Equation 1 and Equation 2, statistical information, Cᵣ (t) and C_{d} (t), are calculated for the same running distance of the in-phase points. It is shown in the step S1-3 in FIG. 2.

Then, the obtained statistical information is analyzed. It is shown in the step S1-4 in FIG. 2. A running distance which a frequency of occurrence in the threshold value ranks at the top rank 1 according to the above determination rule (Equation 3) is output as a main period of the waveform. It is shown in the step S1-5 in FIG. 2.

The following is an application of the invention. Using an example, a heart rate and a respiratory rate which construct main period in the biological signal were detected.

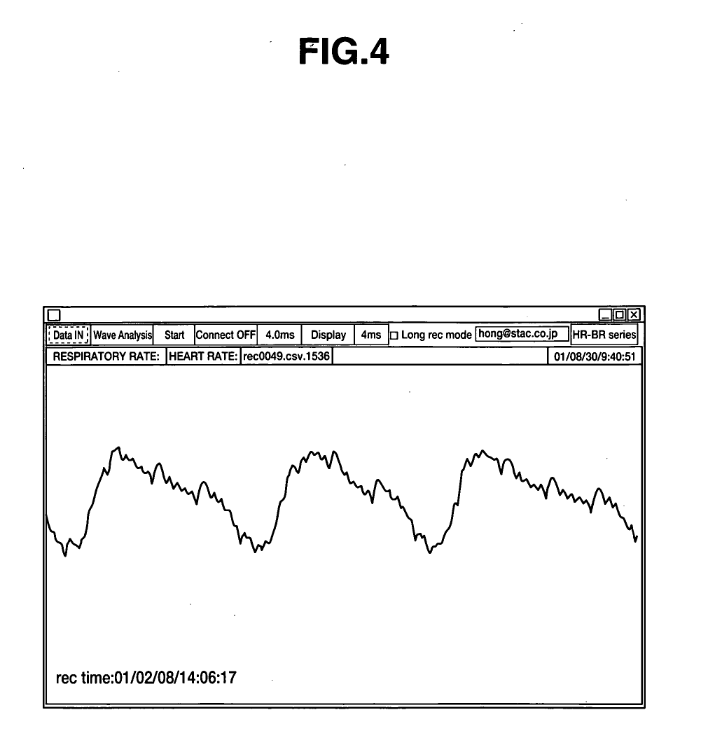

Biological signals in a form of analog signal are detected by biological sensors such as heart beat sensor and respiration sensor. Analog signals of the obtained signals are modulated into digital signals. Then, the digital data are fed into the algorithm. An example of the signals is shown in FIG. 4.

Main periods involved in the signal consist of a heart rate (frequency per minute) and a respiratory rate (frequency per minute).

First, in order to detect a respiratory rate from the input signals, data on the input signals are fed into the algorithm of the invention without modification. With the statistical processing and statistical analysis method of the invention, a respiratory rate as a main period is computed.

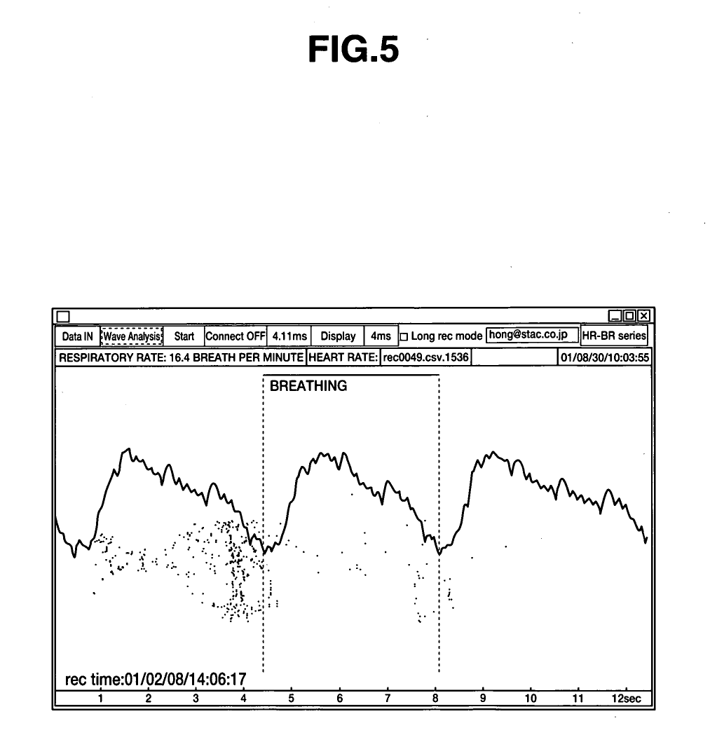

FIG. 5 shows an example of a statistical distribution for the running distance which is an intermediate process.

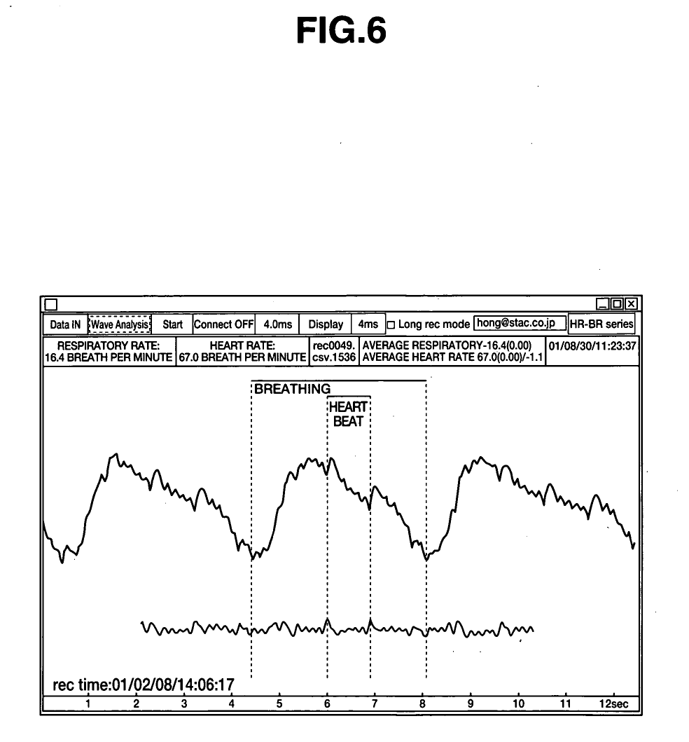

Next, in order to detect a heart rate, components for breathing are removed from the waveform of the input signals by filtering, and then a waveform only with heart rate is produced. FIG. 6 shows the produced waveform of the heart rate.

Then, the produced waveform of heart rate is processed in the same manner as in the case of the respiratory rate so as to produce a heart rate. FIG. 6 shows an example which detects a respiratory rate and a heart rate.

According to the above steps, the invention provides (1) a detection of a waveform period in a stable performance without being influenced by a signal level or a peak height, (2) a compact size measuring device with low power consumption, (3) a precise detection of a waveform period without having the waveform deformed by external factors such as noise and without having a peak position of the waveform fluctuated and (4) a detection period extremely close to a visual waveform period.

### INDUSTRIAL APPLICABILITY

As above explained, a selection of biological signals such as heart beat and breathing and device therefor employs the algorithm of the invention. With the algorithm, a waveform period is detected in a stable performance without being influenced by a signal level or a peak height, and thus a compact-size measuring device with low power consumption can be provided.

Further, the invention provides a precise detection of a waveform period without having the waveform deformed by external factors such as noise and without a peak position of the waveform fluctuated.

Furthermore, the invention provides multiple excellent effects that the conventional measuring methods and devices had difficulty in solving. The effects involve that a detection period extremely close to a visual waveform period can be obtained and so forth.

What is claimed is:

1. A selection of biological signals such as heart beat and breathing, wherein:
forms of biological signals are modulated from time series signals (analog signals) into digital signals with an analog-to-digital modulation means;
a level data of the digital signals is the same
a changing trend (upward trend and downward trend) of the digital signals is the same; and
the most closer two points on an axis of time (in-phase points) are detected with an in-phase points detection means,
wherein after a distance between two in-phase points (running distance between in-phase points) are detected with a detection means of a running distance between in-phase points, a detected information with the detection means of a running distance between in-phase points is computed with a statistical processing means, an information after the statistical processing being analyzed with a measuring means of frequency of occurrence so as to calculate a frequency of occurrence for the same running distance, and a measurement result with the measuring means for frequency of occurrence being analyzed so as to output a most frequent running distance as a main period with a main period output means.

2. A selection device of biological signals such as heart beat and breathing comprising:
an analog-to-digital modulation means which modulates forms of biological signals such as heart beat and breathing from time series signals (analog signals) into digital signals;
an in-phase points detection means, wherein in-phase points are detected on an axis of time, the in-phase points having same level digital signals and same changing trends (upward trend and downward trend);
a detection means of a running distance between in-phase points which detects a distance between two in-phase points;
a statistical processing means which computes the information that the detection means of a running distance between in-phase points detected;
a measuring means of a frequency of occurrence which analyzes the information after statistical processing and measures a frequency of occurrence of the same running distance; and
a main period output means which analyzes the measurement result with the measuring means of a frequency of occurrence and outputs a most frequent running distance as a main period.
